(19) United States
(12) Patent Application Publication
(10) Pub. No.: US 2002/0077568 A1
(43) Pub. Date: Jun. 20, 2002

(54) Biological vessel volume measurement method and apparatus utilizing micro accelerometer

(76) Inventor: Thomas F. Haddock, Ann Arbor, MI (US)

Correspondence Address: John G. Posa, Gifford, Krass, Groh et al, Suite 400, 280 N. Old Woodward Ave., Birmingham, MI, 48009, US

(21) Appl. No.: 09/989,769

(22) Filed: Nov. 20, 2001

Related U.S. Application Data
(60) Provisional application No. 60/252,842, filed on Nov. 22, 2000.

(51) Int. Cl. A61B 5/103

(52) U.S. Cl. 600/587

(57) ABSTRACT

Length and diameter measurements are conducted within an anatomical vessel or body by moving a micro/miniature accelerometer disposed at the distal end of a catheter. The measurements are made by marking an initial position, moving the catheter tip throughout the region, and tracking the position of the tip in real time. As the tip of the catheter moved within the vessel, its position is recorded from the initial (fudicial) position. The linear and/or spatial region within the vessel is then calculated from the accelerometer readings. A three-axis or two-axis approach may be used. The acceleration signals are then processed through double integration to determine the volume of interest, preferably as a computer visualization. The accelerometers used are preferably Micro-Electromechanical System (MEMs) type devices, positioned orthogonally. As the catheter is advanced then pulled back, it moves and strikes the walls of the vessel. The accelerations are recorded and integrated twice, to reveal the path taken by the tip, and subsequently the dimensions of the inside of the vessel volumetrically. The first integration advances from acceleration of velocity, and the next integration, from velocity to distance. The distance is then, in turn, used to determine the shape of the vessel volumetrically in three-space.

### REFERENCE TO RELATED APPLICATIONS

This application claims priority to U.S. provisional patent application Serial No. 60/252,842, filed Nov. 22, 2000, the entire contents of which are incorporated herein by reference.

### FIELD OF THE INVENTION

This invention relates generally to body lumen volume measurement and, in particular, to biological volume measurement apparatus and methods utilizing a catheter with an accelerometer-equipped tip.

### BACKGROUND OF THE INVENTION

Interventional cardiologists most often treat cardiovascular disease through balloon angioplasty and stenting. Stenting reconstructs and supports the artery, and helps maintain patency of the lumen. Nevertheless, restenosis (vessel reocclusion) of coronary arterial lesions previously submitted to balloon angioplasty or angioplasty plus stenting remains a serious medical concern. The incidence of restenosis is about 30 percent after angioplasty alone and 20-30 percent for angioplasty plus stenting.

Pre-deployment measurements of the lesion and adjacent regions of healthy vessel therefore assist greatly in the selection and deployment of a stent. Post-deployment stent diameter measurements are also important in demonstrating that a stent has been adequately deployed. If a stent has not been adequately deployed, the clinician should repeat the deployment until the intended diameter is achieved.

In two controlled multi-center studies, it was found that measurement of the degree of stent deployment by Intravascular Ultrasound (IVUS) improved the outcome. In one study, incomplete deployment of stents was shown to occur in up to 80 percent of patients at nominal pressures (8 to 12 atm), if IVUS was not used. On the other hand, with IVUS, significantly larger minimal stent dimensions were achieved. Achievement of larger stent dimensions was associated with a 44 percent lower rate of restenosis.

In the MULTI-LINK stent trials, the predictors of in-stent restenosis were minimum lumen diameter (MLD), smoking, length of stent, and length of lesion. MLD is a measure of stent deployment, i.e., to what extent the stent was expanded. In addition to assessment of the degree of stent deployment, knowledge of the MLD and lesion length of the vessel being treated permits more accurate stent selection and improved clinical outcomes.

It is estimated that approximately one million coronary balloon angioplasties are performed annually worldwide; 85 percent of these involve the deployment of stents. A significant percentage (20 to 30 percent) of these stents restenose (experience vessel reocclusion), and the patient must be treated again. These procedures cost the U.S. health care providers an estimated $10 billion. Coronary stenting is an additional $1.5 billion industry with an average stent price of over $1,500. Repeat procedures (e.g. laser, rotoblader, brachytherapy, atherectomy) necessitated by restenosis accounts for approximately 20 to 30 percent of these procedures at a cost of an estimated $2.5 billion.

Although IVUS has been shown to be an effective tool in assessing the geometry of deployed stents, the procedure suffers from ergonomic and cost limitations. An IVUS disposable catheter is expensive, currently on the order of $650. The significant IVUS capital equipment expense ($20,000 to $60,000) prevents the unit from being placed in each cath lab. Transporting it from one cath lab to another incurs an additional delay. While some degree of inner-vessel geometry can be obtained either visually, or with quantitative coronary angiography (QCA), these are qualitative measures. QCA analyzes a 2-dimensional image of a 3-dimensional vessel, and is prone to mistakes. Visual estimation via angiography is the least accurate.

Thus, there remains an outstanding need for an accurate yet cost-effective method and apparatus for assessing the inside of a body vessel, ideally including a determination of specific and high-spatial-resolution of lengths, areas and volumes within blood vessels.

### SUMMARY OF THE INVENTION

Broadly according to this invention, length and diameter measurements are conducted within an anatomical vessel or body by moving a micro/miniature accelerometer disposed at the distal end of a catheter. In the preferred embodiment, a three-axis accelerometer sensor is employed, and the axial measurement data is derived from the third accelerometer signal. Alternatively, a two-axis approach may be used, wherein catheter-tip path information in the axial direction is derived from readouts from a pull-back device or other axial-distance measuring device.

The measurements are made by marking an initial position, moving the catheter tip throughout the region, and tracking the position of the tip in real time. As the tip of the catheter moved within the vessel, its position is recorded from the initial (fudicial) position. The linear and/or spatial region within the vessel is then calculated from the accelerometer readings. The acceleration signals are then processed through double integration to render an image of the volume of interest.

The accelerometers used are preferably Micro-Electromechanical System (MEMs) type devices, positioned orthogonally in three dimensions, on the end of the catheter. As the catheter is advanced then pulled back, it moves and strikes the walls of the vessel. The accelerations are recorded and integrated twice, to reveal the path taken by the tip, and subsequently the dimensions of the inside of the vessel volumetrically. The first integration advances from acceleration of velocity, and the next integration, from velocity to distance. The distance is then, in turn, used to determine the shape of the vessel volumetrically in three-space.

The position of the walls of the volume will be known by the greatest extent of the path. In addition, they will be evident by the sharp rises in acceleration as the tip strikes the walls. The magnitudes of these peaks could also serve as indicators of the composition (stiffness) of the walls, which could assist in assessing the state of the vessel. Active or passive approaches may be used to ensure that the sensor tip moves and strikes the walls of the volume being mapped.

The information derived through the invention can serve various useful purposes, including the identification of position and geometrical characteristics of regions of atherosclerotic plaque deposits, as well as measurements beneficial to stent deployment. Use of the invention could significantly reduce restonsis rates by yielding the same geometric information as Intravascular Ultrasound (IVUS). The system and method of the invention should be more convenient than current IVUS systems, with the entire measurement, including the deployment of the sensor and the data processing, taking less than five minutes.

### BRIEF DESCRIPTION OF THE DRAWINGS

FIG. 1A is a drawing which depicts a volume-measuring catheter according to the invention in a retracted state;

FIG. 1B shows the volume-measuring catheter of FIG. 1A in an extended state for use as a probe;

FIG. 2 is an overview showing a system to which the catheter of FIGS. 1A and 1B is interfaced;

FIG. 3 shows how an accelerometer at the tip of the sensing probe describes a semi-helical path along the inner wall of the artery; and

FIG. 4 illustrates a narrowed section of an artery derived through double integration of the accelerometer signals derived through readings associated with the path of FIG. 3.

### DETAILED DESCRIPTION OF THE INVENTION

Accelerometers are known for their ability to provide spatial and dynamic information on systems and objects. The use of accelerometers has been demonstrated in many diverse applications, such as automotive air bags, military guidance systems, even speed and distance monitor for running shoes.

Broadly, this invention uses one or more miniature accelerometers to trace back the path through which it has traveled so as to generate a three-dimensional mapping of a biological vessel. Although the description will concentrate on cardiovascular and coronary arterial applications, it will be apparent to those of skill that the apparatus and method are applicable to other body lumens and volumes, such as peripheral arteries, carotid arteries, renal arteries, pulmonary applications, veins, ducts and glands. Thus, references to “artery” or “vessel” should be taken to include any and all alternative target applications.

Several fundamental accelerometer technologies are applicable to the invention, including open- and closed-loop capacitive devices, and quantum mechanical tunneling devices.

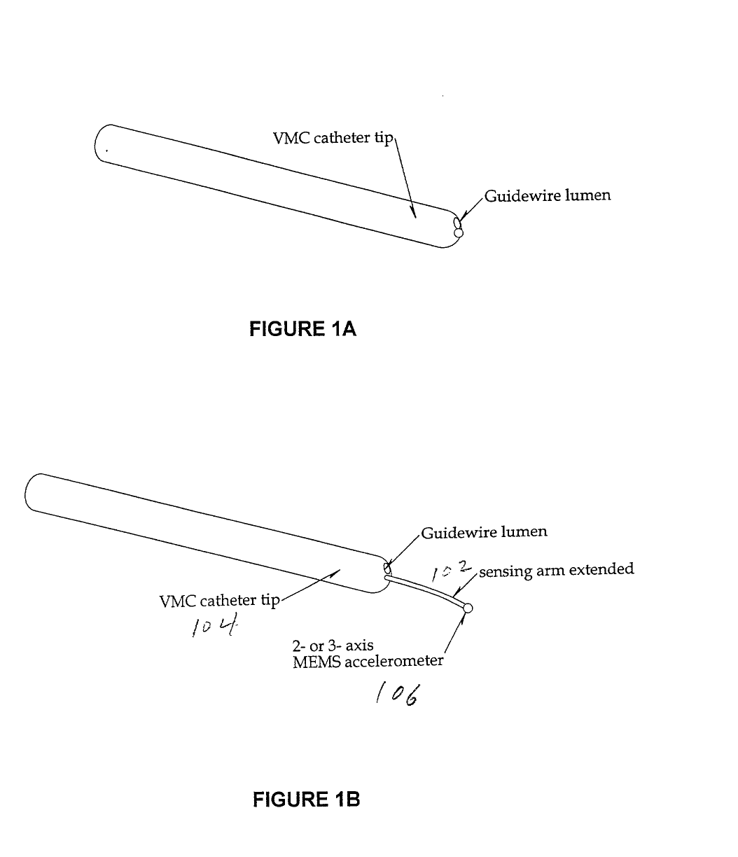

In the preferred embodiment, the sensing element is based on a MEMs (MicroElectroMechanical Systems) accelerometer, measuring acceleration along three orthogonal axes; two orthogonal to the central axis of the catheter, and a third parallel to the central axis of the catheter. As shown in FIG. 1B, a small diameter (preferably on the order of 600-800 μm) cantilevered probe wire 102 (the “sensing probe”) extends from the tip 104 of a catheter.

The accelerometer sensor 106 is located and sealed on the tip of the probe. To produce a biocompatible surface, the sensor to be used at the tip of the sensing probe will either be packaged at the fab level using boron-doped silicon to produce a biocompatible surface, or encapsulated in biocompatible polymers. Since the sensor's signal is acceleration, complete encapsulation of the sensor with a reasonably thin and rigid material will permit adequate signal transmission to the accelerometer while presenting a biocompatible surface.

FIG. 1A shows the probe in a retracted state. Once the catheter is positioned distal to an arterial lesion that is to be mapped, this sensing probe is extended from the tip of the catheter, roughly along the centerline of the catheter. To ensure contact, the sensing probe is slightly curved so that, in its extended configuration, the accelerometer is brought gently against the inner wall of the artery. The sensing probe is then rotated circumferentially with respect to the catheter body (and hence the artery) from controls at the distal end of the catheter, while the catheter is being drawn back along the artery. Active or passive approaches may be used to ensure that the device moves and strikes the vessel walls. For example, a physical configuration may be deployed that forces the tip to gyrate or twist around as it is pulled out. Other techniques may be used to cause the tip to move back and forth and hit the walls as it is extracted.

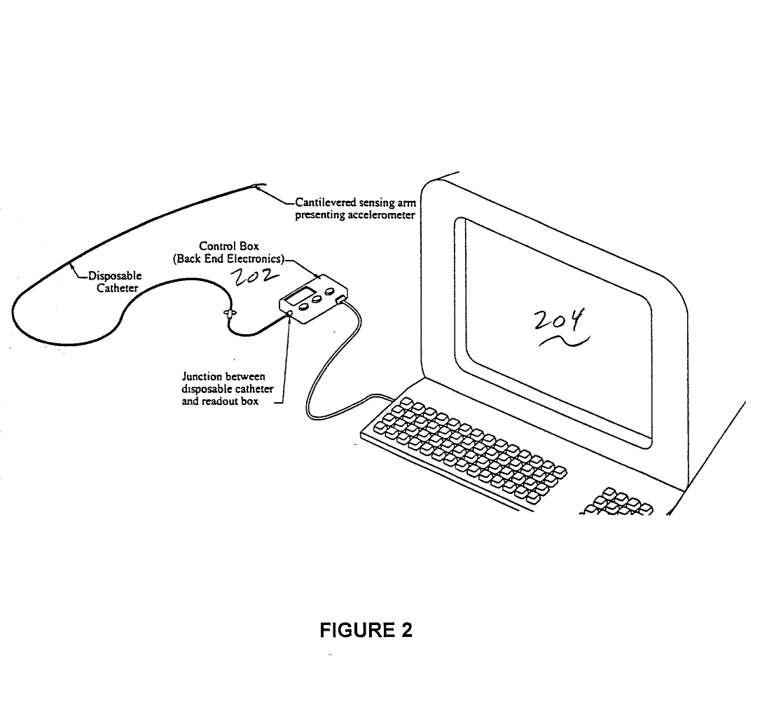

FIG. 2 is an overview showing a system to which the catheter is interfaced. The acceleration signal data is captured in a control unit 202 located at the distal end of the catheter and connected to a personal computer (PC) 204 where the data is analyzed and displayed. A controlling mechanism allows the cardiologist to vary the rotation rate of the probe. During pullback, the data from the two- or three-axes of the acceleration are buffered in the control unit and sent to the PC for processing. This may occur through digitization in the sensor itself, or in the back-end within the control unit. In either case, two or three acceleration signals are double-integrated in the PC to render an image of the volume.

In the preferred embodiment, the entire catheter, with accelerometer, is disposable. The nondisposable unit 202 includes a port to which a line from the catheter connects, making electrical contact for ground and the signal lines from the accelerometer. Note that multiplexing may be used in the catheter to reduce the number of signal wires entering the control unit. The display on the control unit 202 is used to present diagnostics, for example, to determine if the pullback rate is sufficiently low to permit data of a preselected accuracy to be obtained. The data obtained from any pullback is preferably stored in the PC for offline analysis at a later time.

In terms of electronics, the control unit 202 may be based on a central processing unit (CPS) of commercial design, such as a 8-bit or higher-throughput microprocessor, single-chip microcomputer, or custom/semi-custom proprietary processor. The CPU is interfaced to appropriate sample-and-hold circuitry and analog-to-digital converters. Any appropriate software may be used for programming the CPU on a Windows 2000 or Windows NT platform, for example. Since the unit 202 and PC 204 will sit outside the sterile field, such components will not be required to undergo sterilization procedures.

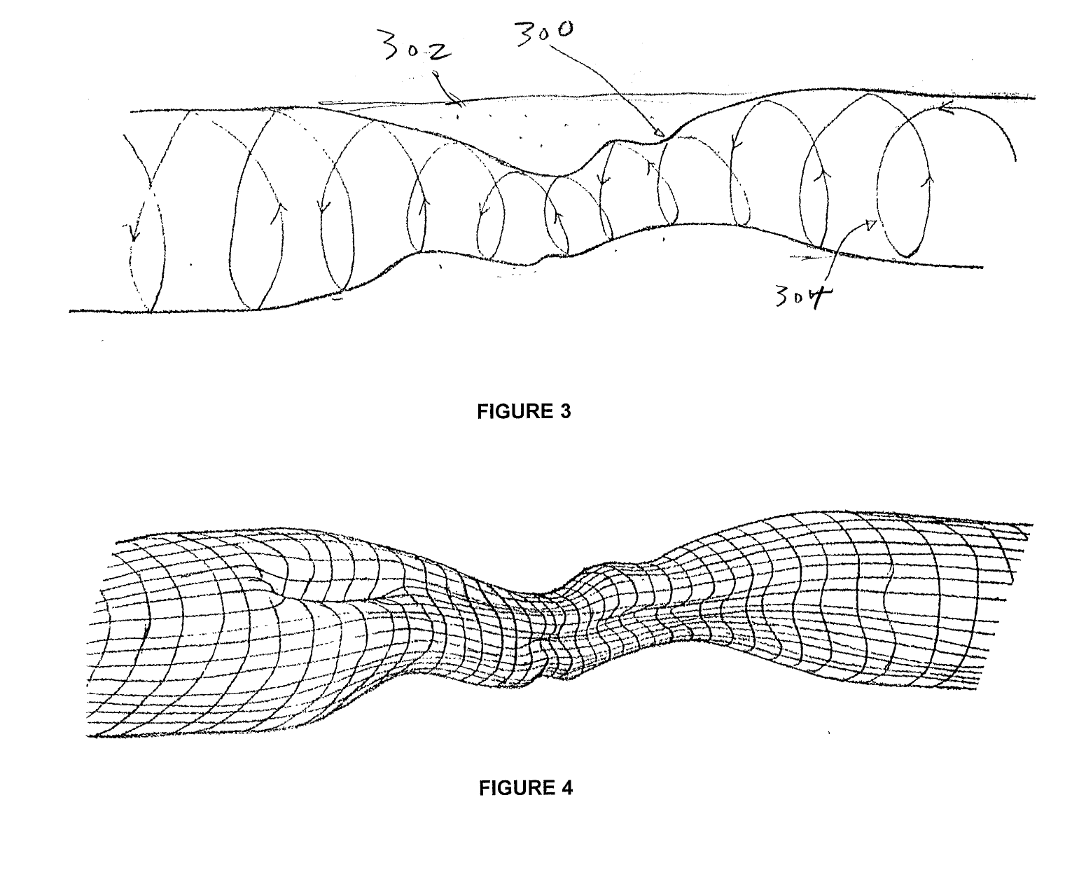

Movement of the probe causes the accelerometer 106 at the tip of the sensing probe to describe a semi-helical path along the inner wall of the artery, under control of the cardiologist, as shown in FIG. 3. The invention is not limited to the tracing of a helical path, however, in that and other volumetric traces may be used, including highly irregular travel, so long as the accelerometer is brought into contact with the wall at enough points to construct an image of the passageway. Of course, the greater the pitch of the helix or other path, the higher will be the final resolution of the 3-D mapping.

Three orthogonal accelerometer signals are preferably taken as the rotating tip is drawn across the area of the vessel, which may include a lesion indicated at 302. Since the measured values will all be relative to the location of the accelerometer when the data flow begins, initial conditions of zero position, velocity and acceleration will be taken at the stationary starting point of the sensing probe tip at the beginning of the pullback.

Software resident on computer 204 uses the raw data to determine the path followed by the accelerometer during the pullback, and subsequently to construct and display a three-dimensional “wire-cage” diagram of the inner volume of the artery. FIG. 4 illustrates an expected result for a narrowed section of artery. This 3-D scaled depiction is generated by double integrating the three orthogonal acceleration values obtained during the pullback of the catheter.

Three-dimensional modeling software is also preferably provided enabling the cardiologist to manipulate this computer diagram of FIG. 4 using a mouse or other pointing device to view the image from any desired angle, enlarge or shrink the image, as well as to view several derived metrics. Such metrics would include displaying a cross-sectional area view of the interior of the artery at any point selected by a pointing device, and displaying distances between any two points selected on the wire-cage diagram. Such a technique would permit any axial distance or any diameter along any circumferential axis to be immediately calculated and displayed, for example, the inner diameter of a deployed stent.

In addition to determining the position of the inner wall of a vessel, the contact accelerometer measurements may also provide information on the modulus of elasticity of the vessel wall. This information might be able to be derived from the relative peak magnitudes of the acceleration signal as the accelerometer contacts the wall. Knowledge of the wall's modulus might be used to classify plaque deposits as hard and calcified or soft and unstable.

The exit point of the sensing probe may be central to the catheter body, or offset to permit a guide wire to pass through the catheter. Spatial resolution of the measurements of the interior of the artery will be a function of draw-back speed and rotation rate of the sensing probe. It is anticipated that draw-back rates on the order of 0.5 mm/second (comparable to IVUS) and rotation rates of the probe of about 1.5 Hz should prove adequate.

In the preferred embodiment, a three-axis accelerometer sensor is employed, and the axial measurement data is derived from the third accelerometer signal. While this is the more desirable design, it requires an additional signal to be taken, perhaps increasing the need for signal multiplexing and requiring a more complex sensor design. This is compounded not only by the addition of a third sensor, but also by the need to produce a sensor on an axis orthogonal to the two radially-oriented axes. Accordingly, a two-axis approach may alternatively be used. In the two-axis design, the two accelerometer axes are orthogonal to each other and to the main axis of the vessel. Measurements of the acceleration signals from these two axes would provide sensing-probe path information in lateral directions, as discussed above, while catheter-tip path information in the axial direction would be derived from readouts from a pull-back device or other axial-distance-measuring device.

The acceleration data is output as a voltage, and these voltages are digitized through a successive approximation analog/digital (A/D) converter under control of the PC using parallel port access with a data acquisition time of approximately 110 μsec. The profile of the channel is obtained from the double integrated data by fitting cubic splines to the points of maximum lateral extent of the deduced path traversed by the accelerometer.

In terms of a specific example, a cardiac coronary arterial catheter according to the invention is approximately 130 to 150 cm long and approximately 1 to 2 mm in diameter. This diameter is on the order of diagnostic catheters such as IVUS or Doppler. Systems under development, and experimental systems are using 3 and 4 Fr catheters. Such small dimensions are well within the precision prototyping and production capabilities of existing commercial suppliers. The sensing probe in this case will preferably feature a diameter of about 300 μm.

As an extension to the basic measurement technique, the invention may be extended to determine to what degree the accelerometer will be able to determine the stiffness of the wall of the channel it is mapping. This will permit an ability to assess biomechanical characteristics of vessel walls. Such information might be useful in classifying plaque deposits as calcified or inflamed based on their modulus of elasticity. Accelerometer pull-back measurements will be made as usual, though the raw acceleration data (not double-integrated to produce position data) will be analyzed to quantify the sensitivity of the acceleration measurements to all modulus.

Various technologies have been employed in micro-accelerometer design. These devices include piezoresistive, piezoelectric, capacitive, quantum mechanical tunneling, resonant, thermal, optical, and electromagnetic (employing, for example, coils instead of capacitive sensing which is also electromagnetic). The preferred design falls into two basic categories; capacitive sensors and quantum mechanical tunneling devices. Capacitive devices are desirable for their high sensitivity, low cost, simple design, good DC response and noise performance, and low power dissipation. Quantum mechanical tunneling devices are desirable for high sensitivity and small size.

A proof mass is suspended from cantilevered proof-mass suspension beams, such that acceleration causes inertial forces of the proof mass of known mass to deflect the mass against the suspension beams. The known (or calibrated) restoring spring forces of the suspension beams permits the proof mass deflection to be translated into a measure of the deflection force and hence the applied acceleration. In the case of capacitive devices, the proof mass is coupled to one or a series of surfaces that move with the proof mass. Proximate to these surfaces are complimentary surfaces fixed to the body of the MEMs device. Circuitry in the device measures the change in capacitance between these two sets of surfaces, and this is the signal. The capacitive devices sense motion of a proof mass suspended from suspension beams under the influence of acceleration of the MEMs device. As an alternate to open-loop devices, closed-loop capacitive force rebalanced designs may also be used, which employ electrostatic attraction to maintain the proof mass in a stationary position to improve dynamic range and linearity.

Quantum mechanical tunneling accelerometers applicable to the invention employ a proof mass suspended by cantilevered suspension beams in the same manner as capacitive accelerometers, and the proof mask is deflected by applied acceleration in the same manner. In these devices, the deflection of the mass against the beams is measured by the variation in tunneling current between two metal electrodes: a tunneling tip electrode rising from the surface of the proof mass and a complimentary tunneling counter-electrode on the body of the device a few Angstroms away. One of the electrodes has a sharp tip, and this geometry enhances electron emission. The tips are brought into within a few Angstroms by the application of an electrostatic field to establish a tunneling current. Movement of the proof mass due to acceleration causes a change in the gap and hence tunneling current. The readout circuit responds by adjusting the deflection voltage. The variation in this voltage is proportional to the applied acceleration.

In use, the invention may be appropriate for use in 50 percent of initial procedures. It is estimated that interventional cardiologists could use the device under the following clinical circumstances: 1) the cardiologist is uncertain of the vessel diameter during angiography due to anomalies that occur when they are looking at a 2-dimensional picture of a 3-dimensional problem; 2) when lesions occur in small vessels (<3.5 mm diameter), or 3) when lesions are long (>15 mm). The interventionalists estimate that the invention would be used to acquire pre-deployment measurements during angiography beginning distal and ending proximal to the lesion. These measurements are extremely valuable in selecting stent size (both length and diameter). After the stent is deployed, the device will be reinserted to confirm that the inner vessel diameter is the size intended by the interventionalist. If use of the invention can reduce repeat procedures by only 10 percent, the potential net savings to third-party insurers might be as great as $250 million annually in the United States alone, as well as immeasurable benefits in terms of human life, health and productivity.

That claimed is:

1. A method of measuring the volume of a biological cavity having an inner wall, comprising the steps of:
moving a multi-axis accelerometer within the cavity such that it interacts with the inner wall at a plurality of points and outputs an acceleration signal indicative of such interactions; and
double integrating the output of the accelerometer to determine the three-dimensional volume of the cavity.

2. The method of claim 1, wherein the axes of the accelerometer are orthogonal to one another.

3. The method of claim 1, wherein the accelerometer is a 3-axis accelerometer.

4. The method of claim 1, wherein the accelerometer is a micro-electromechanical system (MEMs).

5. The method of claim 1, wherein the accelerometer is supported relative to the distal tip of a catheter.

6. The method of claim 5, wherein the accelerometer is mounted on a moveable member facilitating:
a first position, wherein the member is retracted into the tip for insertion into the cavity, and
a second position, wherein the member is extended from the tip for interaction with the inner wall.

7. The method of claim 5, wherein:
the cavity is a human blood vessel; and
the accelerometer interacts with the inner wall as the catheter is withdrawn from the vessel.

8. The method of claim 1, wherein the plurality of points approximates a helix.

9. A method of measuring the volume of a blood vessel having an inner wall, comprising the steps of:
placing a multi-axis accelerometer at the end of a catheter;
inserting the catheter into the blood vessel to be measured;
withdrawing the catheter in such a way that the accelerometer interacts with the inner wall at multiple points and outputs an acceleration signal indicative of such interactions; and
double integrating the output of the accelerometer to determine the three-dimensional volume of the vessel.

10. The method of claim 9, wherein the axes of the accelerometer are orthogonal to one another.

11. The method of claim 9, wherein the accelerometer is a 3-axis accelerometer.

12. The method of claim 9, wherein the accelerometer is a micro-electromechanical system (MBMs).

13. The method of claim 9, wherein the accelerometer is mounted on a moveable member facilitating:
a first position, wherein the member is retracted into the catheter for insertion into the cavity, and
a second position, wherein the member is extended from the catheter for interaction with the vessel wall.

14. The method of claim 9, wherein the plurality of points approximates a helix.

15. A system for measuring the volume of a biological cavity having an inner wall, comprising:
a multi-axis accelerometer operative to output a signal indicative of acceleration as a function of interactions with the inner wall; and
processing circuitry for performing the following functions:
a) receiving the signal output by the accelerometer, and
b) double integrating the signal to determine the three-dimensional volume of the cavity.

16. The system of claim 15, wherein the axes of the accelerometer are orthogonal to one another.

17. The system of claim 15, wherein the accelerometer is a 3-axis accelerometer.

18. The system of claim 15 wherein the accelerometer is a micro-electromechanical system (MEMs).

19. The system of claim 15, further including a catheter having a distal tip, and wherein the accelerometer is supported relative to the distal tip.

20. The system of claim 19, further including a moveable member upon which the accelerometer is mounted, the moveable member facilitating:
a first position, wherein the member is retracted into the tip for insertion into the cavity, and
a second position, wherein the member is extended from the tip for interaction with the inner wall.

21. The system of claim 20, wherein:
the cavity is a human blood vessel; and
the accelerometer interacts with the inner wall as the catheter is withdrawn from the vessel.

22. The system of claim 15, wherein the plurality of points approximates a helix.

23. The system of claim 15, further including apparatus for actively moving the accelerometer to increase the number of interactions.

24. The system of claim 15, wherein the processing circuitry further includes:
a first controller interfaced directly to the accelerometer to perform signal conditioning and direct the accelerometer; and
a computer coupled to the first controller to perform the double integrations.

25. The system of claim 15, wherein the processing circuitry further includes a display for displaying a representatin of the biological cavity in accordance with the result of the double integrations.
